(19) United States
(12) Patent Application Publication
(10) Pub. No.: US 2025/0352262 A1
(43) Pub. Date: Nov. 20, 2025

(54) NEUROSTIMULATION WAVEFORM FOR INCREASED TISSUE ACTIVATION ACROSS VESSEL WALLS

(71) Applicant: Medtronic Ireland Manufacturing Unlimited Company, Dublin 2 (IE)

(72) Inventors: Jerel Keith Mueller, Saint Paul, MN (US); Gerry O. McCaffrey, Galway (IE)

(21) Appl. No.: 19/106,577

(22) PCT Filed: Aug. 28, 2023
(86) PCT No.: PCT/EP2023/073512

§ 371 Date: Feb. 25, 2025

Related U.S. Application Data
(60) Provisional application No. 63/402,716, filed on Aug. 31, 2022.

(51) Int. Cl. A61B 18/14; A61B 18/00; A61B 18/02

(52) U.S. Cl. CPC A61B 18/1492; A61B 18/02; A61B 2018/0016; A61B 2018/0022; A61B 2018/00434; A61B 2018/00642; A61B 2018/00672; A61B 2018/00678; A61B 2018/0072; A61B 2018/00732; A61B 2018/00761; A61B 2018/00767; A61B 2018/00839; A61B 2018/0212; A61B 2018/1467

(57) ABSTRACT

A method of performing and assessing a therapeutic procedure includes navigating a therapeutic device to target tissue, transitioning the therapeutic device from a first, linear configuration to a second, deployed configuration such that a plurality of electrodes on the therapeutic device are in engagement with the target tissue, applying pulses of neurostimulation energy having at least two phases to target tissue via the plurality of electrodes, the neurostimulation energy including an anodal phase and a cathodal phase, wherein a phase of the neurostimulation is switched from anodal to cathodal or cathodal to anodal for each successive pulse, observing a physiological response to the neurostimulation energy indicative of a neural response, denervating the nerves of the target tissue, and applying the neurostimulation energy to the target tissue, wherein a physiological response less than a threshold is indicative of a successful denervation of the nerves of the target tissue.

### TECHNICAL FIELD

The present disclosure relates to systems and methods enabling positioning a therapeutic device within luminal tissues to enhance ablation during a therapeutic procedure. In particular aspects, the present disclosure is directed to methods and systems for denervating nerves in or around vascular tissue.

### BACKGROUND

Catheters have been proposed for use with various medical procedures. For example, a catheter can be configured to deliver neuromodulation (e.g., denervation) therapy to a target tissue site to modify the activity of nerves at or near the target tissue site. The nerves can be, for example, sympathetic or parasympathetic nerves. The sympathetic nervous system (SNS) is a primarily involuntary bodily control system typically associated with stress responses. Chronic over-activation of the SNS is a maladaptive response that can drive the progression of many disease states. For example, excessive activation of the renal SNS has been identified experimentally and in humans as a likely contributor to the complex pathophysiology of arrhythmias, hypertension, states of volume overload (e.g., heart failure), and progressive renal disease.

Percutaneous renal denervation is a minimally invasive procedure that can be used to treat hypertension and other diseases caused by over-activation of the SNS. During a renal denervation procedure, a clinician delivers stimuli or energy, such as radiofrequency, ultrasound, cooling, or other energy to a treatment site to reduce activity of nerves surrounding a blood vessel. The stimuli or energy delivered to the treatment site may provide various therapeutic effects through alteration of sympathetic nerve activity.

### SUMMARY

In accordance with the present disclosure, a method of performing and assessing a therapeutic procedure includes navigating a therapeutic device to target tissue, transition the therapeutic device from a first, linear configuration to a second, helical configuration such that a plurality of electrodes on the therapeutic device are in engagement with the target tissue, applying pulses of neurostimulation energy having at least two phases to the target tissue via the plurality of electrodes, wherein each pulse of the neurostimulation energy includes an anodal phase and a cathodal phase, and a leading phase of the neurostimulation energy is switched from anodal to cathodal or cathodal to anodal for each successive pulse, observing a physiological response to the pulses of neurostimulation energy in excess of a threshold and indicative of a neural response, denervating the nerves of the target tissue, and applying pulses of the neurostimulation energy to the target tissue, wherein a physiological response less than a threshold is indicative of a successful denervation of the nerves of the target tissue.

In aspects, the physiological response may be blood pressure or vessel stiffness.

In other aspects, the target tissue may be one or more of a renal artery, a splanchnic artery, or a hepatic artery.

In certain aspects, during the anodal phase energy may be applied to a first of the plurality of electrodes and received by a second of the plurality of electrodes.

In other aspects, during the cathodal phase energy may be applied to the second of the plurality of electrodes and received by the first of the plurality of electrodes.

In aspects, during the anodal phase or cathodal phase energy may be applied to two or more of the plurality of electrodes or received by two or more of the plurality of electrodes.

In other aspects, the denervation may be achieved by application of monopolar energy to the target tissue via the plurality of electrodes.

In certain aspects, the denervation may be achieved by a therapy modality selected from the group consisting of radiofrequency ablation, microwave ablation, ultrasound ablation, cryoablation, and chemical ablation.

In aspects, the neurostimulation energy may include a frequency of between about 10-30 Hz, a pulse width of between about 2-10 ms, a voltage of between about 5-30 V, and a current of between about 2-500 mA.

In certain aspects, the neurostimulation energy may be multiphasic.

In aspects, the neurostimulation energy may be biphasic or triphasic.

In accordance with another aspect of the present disclosure, a method of assessing a target location for therapy includes applying pulses of neurostimulation to tissue at a target location, wherein the neurostimulation pulses include an anodal phase and a cathodal phase, switching the phases of the neurostimulation pulses from anodal to cathodal or cathodal to anodal for each successive pulse, and observing a physiological response to the neurostimulation pulses, wherein a response in excess of a threshold is indicative of a neural response signifying the target location is a candidate for application of a therapy.

In aspects, the physiological response may be blood pressure or vessel stiffness.

In other aspects, the target location may be one or more of a renal artery, a splanchnic artery, or a hepatic artery.

In certain aspects, during the anodal phase neurostimulation may be applied to a first of a plurality of electrodes and received by a second of the plurality of electrodes.

In other aspects, during the cathodal phase neurostimulation may be applied to the second of the plurality of electrodes and received by the first of the plurality of electrodes.

In aspects, during the anodal phase or cathodal phase neurostimulation may be applied to two or more of the plurality of electrodes or received by two or more of the plurality of electrodes.

In certain aspects, the method may include navigating a diagnostic device to the target location.

In other aspects, the method may include transitioning the diagnostic device from a first configuration to a second configuration such that a plurality of electrodes on the diagnostic device are in engagement with tissue at the target location.

In aspects, the diagnostic device may transition from a linear configuration to a helical configuration to place the electrodes in engagement with tissue at the target location.

In certain aspects, the method may include inflating a balloon to place the electrodes in engagement with tissue at the target location.

In other aspects, the diagnostic device may be a guide catheter having a plurality of electrodes disposed thereon for the delivery of the neurostimulation to the target tissue.

In aspects, the method may include placing at least two of the plurality of electrodes of the guide catheter into engagement with the tissue at the target location.

In other aspects, the method may include applying denervation therapy to the target tissue, the denervation therapy selected from the group consisting of radiofrequency ablation, microwave ablation, ultrasound ablation, cryoablation, and chemical ablation.

In certain aspects, the neurostimulation energy may be multiphasic.

In aspects, the neurostimulation energy may be biphasic or triphasic.

In accordance with another aspect of the present location, a system for performing a diagnostic and therapeutic procedure includes a catheter including a plurality of electrodes and configured for placement proximate target tissue, and an energy source operably coupled to the catheter, the energy source having a diagnostic mode, wherein pulses of neurostimulation energy are generated for delivery between at least two of the plurality of electrodes in a bipolar manner, the pulsed neurostimulation energy including an anodal phase and a cathodal phase, and the energy source configured to switch the phases of the pulsed neurostimulation energy from anodal to cathodal or cathodal to anodal for each successive pulse, and a denervation mode, wherein monopolar energy is generated for delivery by the plurality of electrodes for denervation of nerves of the target tissue.

In aspects, the catheter may have a first configuration and a second configuration, wherein in the second configuration the plurality of electrodes on the catheter are in engagement with tissue at the target tissue.

In certain aspects, the first configuration may be a linear configuration for navigation to the target tissue and the second configuration is a helical configuration to place the plurality of electrodes in engagement with tissue at the target tissue.

In other aspects, the system may include a balloon disposed on the catheter, wherein inflation of the balloon places the plurality of electrodes in engagement with tissue at the target tissue.

In accordance with another aspect of the present disclosure, a system for performing a diagnostic and therapeutic procedure includes a catheter including a plurality of electrodes and configured for placement proximate target tissue, an energy source operably coupled to the catheter, the energy source generating pulses of neurostimulation for delivery between at least two of the plurality of electrodes in a bipolar manner, the pulsed neurostimulation energy including anodal and cathodal phases, and the energy source configured to switch the phases of the pulsed neurostimulation energy from anodal to cathodal or cathodal to anodal for each successive pulse, and a therapy source coupled to the catheter for delivery of denervation therapy for denervation of nerves of the target tissue.

In aspects, the therapy source may be selected from the group consisting of a cryogenic source for delivery of cryogenic medium, an RF generator for generating monopolar radio-frequency energy, a microwave generator for generating microwave energy, and a chemical source for delivery of a chemical medium.

In aspects, the system may include a balloon disposed on the catheter, wherein inflation of the balloon places the plurality of electrodes in engagement with tissue at the target tissue.

In certain aspects, the therapy source may be a cryogenic source for delivery of cryoablation medium, wherein the balloon is be inflated with the cryoablation medium.

In other aspects, the energy source may be integrated with the therapy source.

In certain aspects, the catheter may include a second plurality of electrodes configured for placement proximate target tissue, the second plurality of electrodes coupled to the therapy source for delivery of denervation therapy to the target tissue.

In aspects, the energy source and the therapy source may each be coupled to the plurality of electrodes, such that the plurality of electrodes delivery neurostimulation energy to the tissue in a diagnostic mode and denervation therapy to the tissue in a denervation mode.

In certain aspects, the catheter may have a first configuration and a second configuration, wherein in the second configuration the plurality of electrodes on the catheter are in engagement with tissue at the target tissue.

In other aspects, the first configuration may be a linear configuration for navigation to the target tissue and the second configuration is a helical configuration to place the plurality of electrodes in engagement with tissue at the target tissue.

In accordance with another aspect of the present disclosure, a system for performing a diagnostic and therapeutic procedure includes a catheter including a plurality of electrodes and configured for placement proximate target tissue, and a workstation operably coupled to the catheter, the workstation including a memory and a processor, the memory storing instructions, which when executed by the processor cause the processor to apply pulses of neurostimulation energy having at least two phases to the target tissue via the plurality of electrodes, wherein each pulse of the neurostimulation energy includes an anodal phase and a cathodal phase, and a phase of the neurostimulation energy is switched from anodal to cathodal or cathodal to anodal for each successive pulse, observe a physiological response to the pulses of neurostimulation energy in excess of a threshold and indicative of a neural response, denervate the nerves of the target tissue, and apply pulses of the neurostimulation energy to the target tissue, wherein a physiological response less than a threshold is indicative of a successful denervation of the nerves of the target tissue.

In aspects, the system may include an energy source operably coupled to the catheter, the energy source configured to generate a therapy modality selected from the group consisting of radiofrequency ablation, microwave ablation, ultrasound ablation, cryoablation, and chemical ablation.

In other aspects, the energy source may be configured to generate the neurostimulation energy when in a diagnostic mode and generate a therapy modality when in a denervation mode.

In certain aspects, the neurostimulation energy may include a frequency of between about 10-30 Hz, a pulse width of between about 2-10 ms, a voltage of between about 5-30 V, and a current of between about 2-500 mA.

In other aspects, the system may include a balloon disposed on the catheter, wherein inflation of the balloon places the plurality of electrodes in engagement with the target tissue.

Further disclosed herein is a method of performing and assessing a therapeutic procedure includes navigating a therapeutic device to target tissue, transitioning the therapeutic device from a first, linear configuration to a second, deployed configuration such that a plurality of electrodes on the therapeutic device are in engagement with the target tissue, applying pulses of neurostimulation energy having at least two phases to target tissue via the plurality of electrodes, the neurostimulation energy including an anodal phase and a cathodal phase, wherein a phase of the neurostimulation is switched from anodal to cathodal or cathodal to anodal for each successive pulse, observing a physiological response to the neurostimulation energy indicative of a neural response, denervating the nerves of the target tissue, and applying the neurostimulation energy to the target tissue, wherein a physiological response less than a threshold is indicative of a successful denervation of the nerves of the target tissue.

### BRIEF DESCRIPTION OF THE DRAWINGS

Various aspects and embodiments of the disclosure are described hereinbelow with references to the drawings, wherein:

FIG. 1 is a schematic diagram of a therapy system provided in accordance with the present disclosure;

FIG. 2 is a schematic view of a workstation of the therapy system of FIG. 1;

FIG. 3 is a perspective view of a therapeutic device of the therapy system of FIG. 1;

FIG. 4 is a perspective view of the therapeutic device of FIG. 3 shown advanced within a portion of the patient's anatomy and in a deployed condition;

FIG. 5 is a perspective view of another embodiment of a therapeutic device of the therapy system of FIG. 1 provided in accordance with the present disclosure; and

FIG. 6 is a flow chart illustrating a method of performing a therapeutic procedure in accordance with the present disclosure.

### DETAILED DESCRIPTION

This disclosure is directed to therapeutic systems and methods for denervation or neuromodulation of nerves such as the sympathetic, or in certain embodiments, parasympathetic, nerves, and in particular, unmyelinated nerve fibers in and around blood vessels and other luminal tissues. To enhance the efficacy of denervating the nerves, the therapeutic system is configured to apply neurostimulation to blood vessels or other luminal tissues having a multiphasic-pulsed waveform (e.g., biphasic, triphasic, etc.). In one non-limiting embodiment, and as generally described herein, the neurostimulation includes a biphasic waveform, with each pulse of the biphasic waveform having an anodal leading phase and a cathodal trailing phase or vice versa. In at least one embodiment the therapeutic system is configured to alternate the leading phase of each pulse of the biphasic waveform during the application of the neurostimulation such that, for example, a first pulse includes an anodal leading phase and a cathodal trailing phase, a subsequent, second pulse includes a cathodal leading phase and an anodal trailing phase, and a subsequent, third pulse returns to an anodal leading phase and a cathodal trailing phase. The leading phase of each pulse of the biphasic waveform is alternated for the duration of the application of the neurostimulation. As a result, a neural response to the neurostimulation is enhanced as compared to continuous first phase biphasic waveforms and monophasic waveforms as is known in the art. This in turn increases the likelihood of stimulating neural tissue and decreases the amount of time required to identify suitable neural tissue for denervation therapy. Further application of neurostimulation promotes accurate determinations of the suitability of a location for receiving therapy since a greater amount of neural tissue is stimulated by the alternating biphasic waveform described herein. Although generally described throughout the present disclosure as having a biphasic waveform, as noted hereinabove, the neurostimulation applied to the tissue may include any multiphasic waveform in which one or more of the phases are inverted for each successive pulse without departing from the scope of the present disclosure.

The therapeutic devices contemplated in this disclosure can apply one or more of a variety of therapeutic modalities. For example, the therapeutic modalities considered within the scope of this disclosure include monopolar or bipolar radiofrequency, microwave, cryogenic, ultrasound, chemical, and other yet to be developed modalities. Any of these therapy modalities may be incorporated into a therapeutic device, such as a catheter, that is configured for navigation to a desired location within the patient. A catheter configured to delivery one or more of these therapeutic modalities may be percutaneously navigated, for example via the femoral artery, to reach the blood vessels of the aorta including the celiac artery, hepatic arteries, splanchnic arteries, mesenteric arteries and others that are enervated with sympathetic nerves or are proximate one or more sympathetic nerve ganglia. Such a catheter may also be laparoscopically placed in one or more of the above-identified blood vessels, or another luminal tissue without departing from the scope of the present disclosure.

The therapeutic device described herein are also configured to deliver neurostimulation to the blood vessel or other luminal tissue. This neurostimulation can take a variety of forms (e.g., magnitude, frequency, duration), however, the neurostimulation signal itself is a multi-phasic pulsed waveform (e.g., biphasic or triphasic pulses). The amplitude, frequency, pulse width, and/or duration of the neurostimulation can be selected and/or modified to ensure stimulation of the target nerves of the periluminal tissue (e.g., unmyelinated nerve fibers) without damaging the luminal tissue or the nerves within or surrounding the luminal tissue or causing excess vasoconstriction about the therapeutic device (e.g., inhibiting the movement of the therapeutic device within the luminal tissue).

As described hereinabove, one of the goals of the neurostimulation is to identify the blood vessel or luminal tissue portions that are proximate to sympathetic nerves that are candidates for denervation therapy. As such, a physiological response to the application of neurostimulation to the luminal tissue is measured and/or observed, such as an increase in blood pressure or an increase in luminal tissue stiffness. If no or insufficient response is observed, the position of the therapeutic device within the vessel or luminal tissue may be adjusted, for example, proximal or distal, or clockwise or anti-clockwise, and the neurostimulation once again applied and the physiological response again measured and/or observed. The physiological response to the neurostimulation can be compared to one or more predetermined thresholds, such that a blood pressure or tissue stiffness exceeding the threshold is indicative of tissue that is a candidate for denervation whereas a blood pressure or tissue stiffness falling below the threshold is indicative of tissue that may not be a candidate for denervation. In embodiments, neurostimulation may be applied at various locations and/or orientations within the vessel or luminal tissue and the physiological response measured and recorded at each location. The position and/or orientation where the physiological response is the greatest can be selected as the position and/orientation where denervating would be most effective.

The neurostimulation may be delivered to the vessel or luminal tissue via two or more energy delivery elements or electrodes disposed in spaced relation to one another. These electrodes may be disposed on the outer surface of a therapy catheter, on a balloon, on a separate therapeutic device, such as a guide catheter, guide wire, etc. The electrodes may deliver neurostimulation to the vessel or luminal tissue independently of one another such that during the anodal phase of the biphasic pulse, the neurostimulation is applied to the vessel via a first of the electrodes and received by a second electrode and during the cathodal phase of the biphasic pulse the neurostimulation is applied to the target tissue via the second electrode and received by the first electrode. In embodiments, during the anodal phase or the cathodal phase of the bipolar pulse, the neurostimulation is applied by two or more of the electrodes or received by two or more of the electrodes.

The therapeutic device may be coupled to a therapy source and a neurostimulation source, although it is envisioned that the therapy source and the neurostimulation source may be the same and capable of generating both therapy and neurostimulation. For example, an electrical generator may be configured to generate biphasic pulses to be supplied to the neurostimulation applying portions of the therapeutic device and the supply monopolar RF energy to the therapy applying portions, although it is contemplated that the neurostimulation applying portions and the therapy applying portions of the therapeutic device may be one in the same, such that neurostimulation and monopolar RF energy may be applied to the tissue via the same electrodes at different points in time. Additionally, or alternatively, the modalities for therapy and neurostimulation may be similar, as described hereinabove, or very different, such as biphasic neurostimulation in combination with cryoablation therapy. Any combination of the above-described therapy modalities and neurostimulation modalities is contemplated within the scope of the present disclosure.

In accordance with aspects of the present disclosure, the therapeutic device may be navigated within the vessels or luminal tissue in one configuration (e.g., a linear configuration) and once located at a desired location, deployed or otherwise actuated to achieve a second configuration (e.g., an expanded balloon, deployed needles, shape memory helical shape, etc.). The second configuration may be achieved either before or after application of the neurostimulation. Regardless of when applied, in accordance with the present disclosure, the application of the neurostimulation may be performed multiple times before, during, and after the application of therapy. As such, the application of the neurostimulation may also be employed to assess the effectiveness of the denervation procedure. Following a successful denervation, the application of neurostimulation may result in limited to no physiological response as the nerves which stimulate the physiological response have been severed and are unable to trigger any such response.

For ease of description, much of the following description focuses on implementations of electrical stimulation and RF denervation. Those having skill in the art will recognize that the methods and systems described herein may employ any of the therapy and/or neurostimulation modalities described herein. Similarly, the following description focuses on navigation to and application of neurostimulation and/or therapy to the renal artery to denervate sympathetic or, in certain embodiments, parasympathetic, nerves in, around, and proximate the renal arteries. However, the present disclosure is not so limited and can be employed for denervating nerves accessible via any blood vessel described herein of other luminal tissue (e.g., a bile duct).

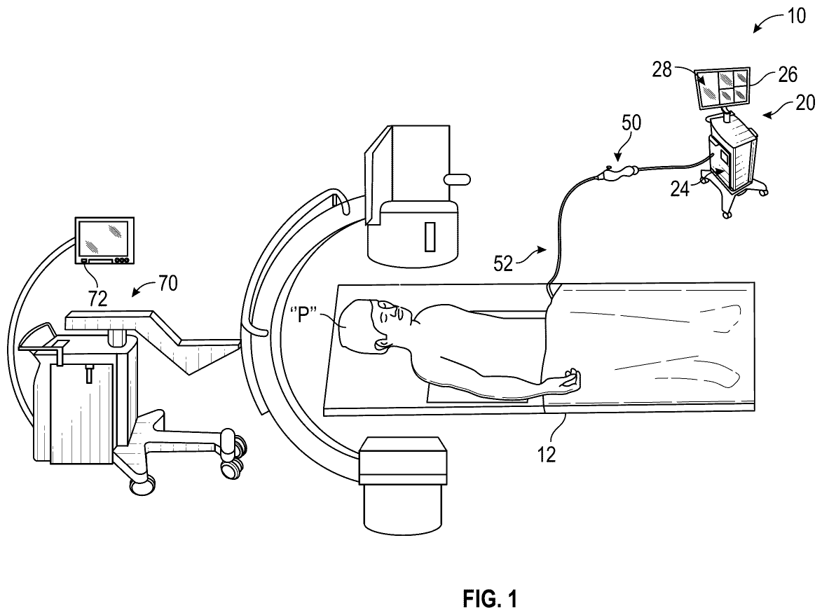

Turning now to the drawings, FIG. 1 illustrates a therapy system provided in accordance with the present disclosure and generally identified by reference numeral 10. As will be described in further detail hereinbelow, the therapy system 10 enables navigation of a therapeutic device 50 to a desired location within the patient's anatomy (e.g., the patient's renal artery), delivery of neurostimulation to tissue within the renal artery, observing a physiological response to the application of neurostimulation to the tissue, adjustments of a position of the therapeutic device within the renal artery based upon the physiological response, reapplication of the neurostimulation to the tissue at the adjusted position, application of denervation therapy to the tissue within the renal artery to denervate sympathetic nerves within the tissue, and delivery of neurostimulation to the denervated tissue observe the physiological response to the neurostimulation and assess the efficacy of the denervation therapy.

The therapy system 10 includes a workstation 20, a therapeutic device 50 operably coupled to the workstation 20, and an imaging device 70, which may be operably coupled to the workstation 20. The patient “P” is shown lying on an operating table 12 with the therapeutic device 50 inserted through a portion of the patient's femoral artery, although it is contemplated that the therapeutic device 50 may be inserted into any suitable portion of the patient's vascular network that is in fluid communication with a desired blood vessel for therapy. Although generally described as having one therapeutic device 50, it is envisioned that the therapeutic system 10 may employ any suitable number of therapeutic devices 50. The therapeutic devices 50 may employ the same or different therapy modalities may and be operably coupled to the workstation 20. Further, the therapeutic device 50 may employ a guidewire 64 or a guide catheter 62 without departing from the scope of the disclosure.

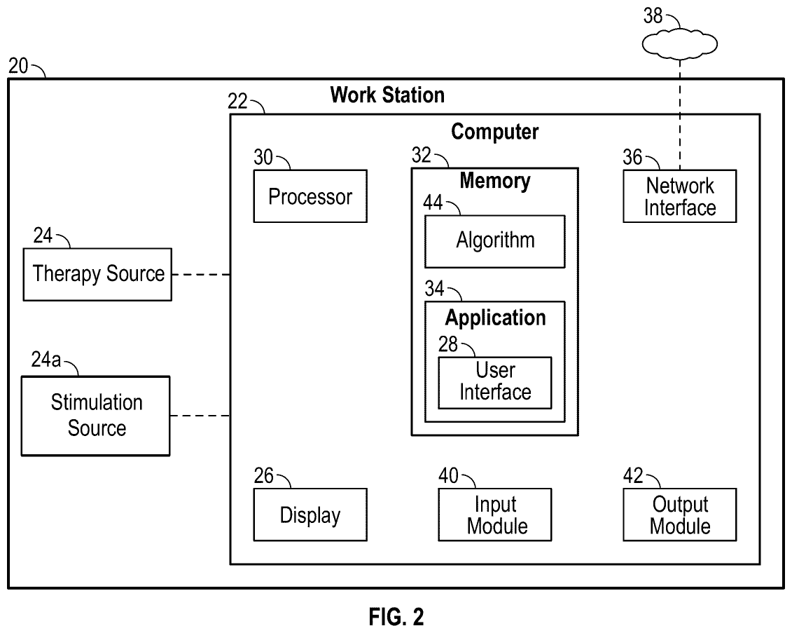

Continuing with FIG. 1 and with additional reference to FIG. 2, the workstation 20 includes a computer 22, a therapy source 24 (e.g., an RF generator, a microwave generator, an ultrasound generator, a cryogenic medium source, a chemical source, etc.) operably coupled to the computer 22, and a neurostimulation source 24a operably coupled to the computer 22. Although generally described as being separate from the therapy source 24, it is envisioned that the stimulation source 24a may be integrated within the therapy source 24, as described hereinabove, and the therapy source 24 may generate both therapy and neurostimulation modalities.

The computer is coupled to a display 26 that is configured to display one or more user interfaces 28. The computer 22 may be a desktop computer or a tower configuration with display 26 or may include a laptop computer or other computing device. The computer 22 includes a processor 30 which executes software stored in a memory 32. The memory 32 may store one or more applications 34 and/or algorithms 44 to be executed by the processor 30. A network interface 36 enables the workstation 20 to communicate with a variety of other devices and systems via the internet. The network interface 36 may connect the workstation 20 to the Internet via a wired or wireless connection. Additionally, or alternatively, the communication may be via an ad hoc Bluetooth® or wireless network enabling communication with a wide-area network (WAN) and/or a local area network (LAN). The network interface 36 may connect to the Internet via one or more gateways, routers, and network address translation (NAT) devices. The network interface 36 may communicate with a cloud storage system 38, in which further data, image data, and/or videos may be stored. The cloud storage system 38 may be remote from or on the premises of the hospital such as in a control or hospital information technology room. It is envisioned that the cloud storage system 38 could also serve as a host for more robust analysis of acquired images (e.g., fluoroscopic, computed tomography (CT), magnetic resonance imaging (MRI), cone-beam computed tomography (CBCT), etc.), data, etc. (e.g., additional or reinforcement data for analysis and/or comparison). An input module 40 receives inputs from an input device such as a keyboard, a mouse, voice commands, an energy source controller (e.g., a foot pedal or handheld remote-control device that enables the clinician to initiate, terminate, and optionally, adjust various operational characteristics of the therapy source 24 and/or neurostimulation source 24a, including, but not limited to, power delivery), amongst others. An output module 42 connects the processor 30 and the memory 32 to a variety of output devices such as the display 26. In embodiments, the display 26 may be a touchscreen display.

The therapy source 24 generates and outputs one or more of RF energy (monopolar or bipolar), microwave energy, ultrasound energy, cryogenic medium, or chemical ablation medium via an automated control algorithm 44 stored on the memory 32 and/or under the control of a clinician. As can be appreciated, the therapy generated or output by the therapy source 24 changes a temperature of the tissue (e.g., increases or decreased the temperature) to achieve the desired denervation of the nerves. The therapy source 24 may be configured to produce a selected modality and magnitude of energy and/or therapy for delivery to the treatment site via the therapeutic device 50, as will be described in further detail hereinbelow. The therapy source 24 may monitor voltage and current applied to target tissue via the therapeutic device 50 and monitors the temperature of the target tissue or tissue proximate the target tissue, and/or a portion of the therapeutic device 50.

The neurostimulation source 24a generates neurostimulation having a biphasic waveform and an energy level that is less than the therapy generated by the therapy source such that the neurostimulation generated by the neurostimulation source 24a does not denervate the target tissue. Rather, the neurostimulation source 24a generates neurostimulation capable of effectuating a response from the nerves indicative of tissue that would be a candidate for denervation, such as an increase in blood pressure, an increase in vessel stiffness, pulse wave velocity, augmentation pressure, heart rate variability, etc. The neurostimulation source 24a generates a biphasic waveform where a leading phase of each successive pulse of the biphasic waveform is switched or otherwise inverted. In this manner, a biphasic waveform having an initial pulse with an anodal leading phase and a cathodal trailing phase will be followed by a second pulse with a cathodal leading phase and an anodal trailing phase which will be followed by a third pulse returning to an anodal leading phase and a cathodal trailing phase, and so on. Alternatively, a biphasic waveform having an initial pulse with a cathodal leading phase and an anodal trailing phase will be followed by a second pulse with an anodal leading phase and a cathodal trailing phase which will be followed by a third pulse returning to a cathodal leading phase and an anodal trailing phase. As can be appreciated, the leading phase of each pulse of the biphasic waveform is alternated for the duration of the application of neurostimulation to the target tissue.

As noted above, the amplitude, frequency, pulse width, and/or duration of the neurostimulation can be selected and/or modified to ensure neurostimulation of the sympathetic nerves of the luminal tissue without damaging the luminal tissue or the nerves within or surrounding the luminal tissue or causing excess vasoconstriction about the therapeutic device (e.g., inhibiting the movement of the therapeutic device within the luminal tissue). As will be appreciated, a pulse duration (pulse width) of the biphasic pulses may be modified to ensure that anodic stimulation of the tissue is maintained as at certain pulse durations regions of anodic stimulation may dissipate or otherwise disappear resulting in fewer regions of neurostimulation. In one non-limiting embodiment, the neurostimulation source 24a generates biphasic waveforms having a frequency of between approximately 10-30 Hz, a voltage of between approximately 5-30 V, a current of between approximately 2-500 mA, and a pulse width of between approximately 2-10 ms. It is envisioned that in embodiments where unmyelinated nerve fibers are targeted, the pulse width of the biphasic waveform may be between approximately 10-120 ms.

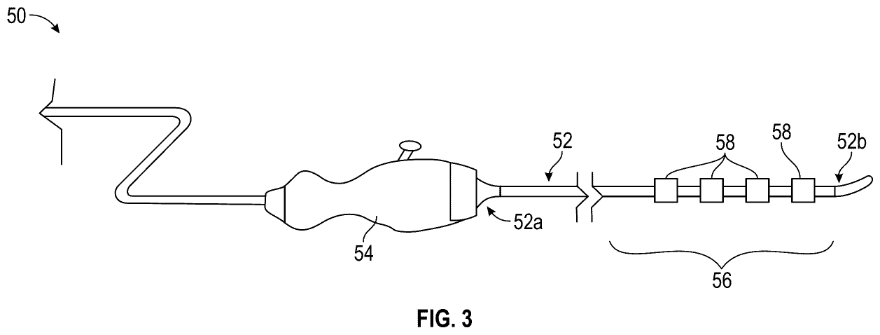

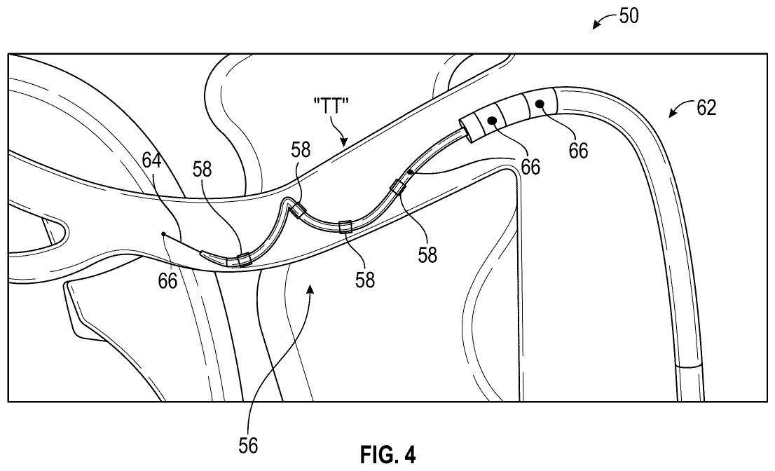

FIGS. 3 and 4 depict one embodiment of a therapeutic device 50 in accordance with the present disclosure. The therapeutic device 50 includes an elongated shaft 52 having a handle 54 disposed on a proximal end portion 52a of the elongated shaft 52. The therapeutic device 50 includes a therapeutic assembly 56 at which one or more therapy electrodes 58 are located. The elongated shaft 52 of the therapeutic device 50 is configured to be advanced within a portion of the patient's vasculature, such as a femoral artery or other suitable portion of patient's vascular network that is in fluid communication with the patient's renal artery. In embodiments, the therapeutic assembly 56 is configured to be transformed from an initial, undeployed configuration having a generally linear profile (FIG. 3), to a second, deployed or expanded configuration, where the therapeutic assembly 56 forms a generally spiral and/or helical configuration (FIG. 4) for delivering energy at the treatment site and providing therapeutically-effective electrically and/or thermally induced renal neuromodulation. In this manner, when in the second, expanded configuration, the therapeutic assembly 56, and in particular, the individual electrodes 58, is pressed against or otherwise contacts the walls of the patient's vasculature tissue. Although generally described as transitioning to a spiral and/or helical configuration, it is envisioned that the therapeutic assembly 56 may be deployed in other configurations without departing from the scope of the present disclosure. Further, the therapeutic device 50 may be configurable, for example, using one or more pull wires (not shown) to adjust the configuration to promote contact between the electrodes 58 and the wall of the renal artery. As such, the therapeutic device 50 may be capable of being placed in one, two, three, four, or more different configurations depending upon the design needs of the therapeutic device 50 or the location at which therapy is to be applied.

As depicted in FIG. 4, the elongated shaft 52 may be configured to be received within a portion of a guide catheter or guide sheath (such as a 6F guide catheter) 62 that is utilized to navigate the therapeutic device 50 to a desired location at which point if a guide catheter 62 is retracted to uncover the therapeutic portion 56 of the therapeutic device 50. As noted hereinabove, retraction of the guide catheter 62 may enable the therapeutic portion 56 to transition from the first, undeployed configuration, to the second, deployed or expanded configuration.

The elongated shaft 52 of the therapeutic device 50 may further include an aperture (not shown) that is configured to slidably receive a guidewire 64 over which the therapeutic device 50, either alone or in combination with the guide catheter 62, are advanced. In this manner, the guidewire 64 is utilized to guide the therapeutic device 50 to the target tissue using over-the-wire (OTW) or rapid exchange (RX) techniques, at which point the guide wire may be partially or fully removed from the therapeutic device 50, enabling the therapeutic device 50 to transition from the first, undeployed configuration (FIG. 3), to the second, deployed or expanded configuration (FIG. 4). As noted elsewhere herein, the therapeutic device 50 may be transition from the first, undeployed configuration to the second, deployed configuration automatically (e.g., via a shape memory alloy, etc.) or manually (e.g., via pull wires, guide wire manipulation, etc. that is controlled by the clinician).

Continuing with FIGS. 3 and 4, in embodiments where the therapeutic device 50 is an RF ablation catheter, the therapeutic portion 56 includes one or more electrodes 58 disposed on an outer surface thereof that are configured to contact a portion of the patient's vascular tissue when the therapeutic device 50 is placed in the second, expanded configuration. As shown herein, the therapeutic device 50 includes four electrodes 58. However, the present disclosure is not so limited and the therapeutic device 50 may have more or fewer electrodes 58 without departing from the scope of the present disclosure. One of skill in the art will recognize that the electrodes 58 may be replaced with ultrasound transducers, microwave antennae, ports for delivery of cryoablation medium or chemical medium and other implements and/or ablation and denervation modalities without departing from the scope of the present disclosure.

As illustrated in the figures, the electrodes 58 are disposed in spaced relation to one another along a length of the therapeutic device 50 forming the therapeutic portion 56. As will be appreciated, these electrodes 58 are in communication with the therapy source 24 which produces, for example, monopolar RF energy to denervate the sympathetic nerves of the relevant blood vessel. Additionally, or alternatively, the electrodes 58 may delivery RF energy independently of one another (e.g., monopolar), simultaneously, selectively, sequentially, and/or between any desired combination of the electrodes 58 (e.g., bipolar). It is envisioned that the therapy source 24 is also the neurostimulation source 24a and includes a diagnostic mode, where the therapy source 24 generates neurostimulation having a biphasic waveform described in accordance with the present disclosure, and a denervation mode, where the therapy source 24 generates RF energy to denervate the nerves of the relevant blood vessel. It is contemplated that the therapy source 24 may be manually switched from diagnostic mode to denervation mode and vice versa or may be automatically switched by an algorithm 44 stored on the memory 32 of the computing device. In at least one aspect of the present disclosure, the electrodes 58 are in communication with a stand-alone neurostimulation source 24a to deliver neurostimulation to the blood vessel in question. The neurostimulation signal (e.g., the biphasic waveform), is generated by the neurostimulation source 24a and communicated to the electrodes 58 causing stimulation of the sympathetic nerves as described herein.

In embodiments, during the anodal phase of the biphasic pulse, the neurostimulation is applied to the target tissue via a first of the electrodes 58 and received by a second of the electrodes 58 in a bipolar manner and during the cathodal phase of the biphasic pulse the neurostimulation is applied to the target tissue via the second of the electrodes 58 and received by the first of the electrodes 58 in a bipolar manner. It is envisioned that during the anodal phase or the cathodal phase of the bipolar pulse, the neurostimulation is applied by two or more of the electrodes 58 or received by two or more of the electrodes 58 in any suitable configuration, such as a proximal most electrode 58 and a distal most electrode 58, a proximal most electrode 58 and a next proximal most electrode 58, a proximal most electrode 58 and an electrode 58 disposed just proximal of the distal most electrode 58, etc.

Further, one or more algorithms 44 may be employed for the stimulation of the multiple electrodes 58. Where for example, if there are four electrodes, there is a firing order for the electrodes 58 to apply the neurostimulation. In such an example the electrodes 58 may connect in a bipolar fashion as follows. In a first anodal phase between a first electrode and a fourth, first cathodal phase between the fourth electrode and the first electrode. This may be followed by a second cathodal phase between the fourth electrode and the first electrode and a second anodal phase between the first electrode and the fourth electrode. This may be followed in a similar manner by different pairs of electrodes 58, for example between the first and third electrodes 58, the first and second electrodes 58. A similar pattern may be followed between second and fourth electrodes and the second and third electrodes. Still further, an anodal and cathodal phase need not be between the same pairs of electrodes. For example, a first anodal phase may be between a first and a fourth electrode and be followed by a cathodal phase between the fourth and the second electrode. Alternatively, the first anodal phase may be between a first and a fourth electrode and followed by a cathodal phase between the fourth and first electrodes 58, as in the first example, however the second anodal phase may be between the second and the fourth electrodes followed by a second cathodal phase between the fourth and second electrodes. The firing order of the electrodes 58 is limited only by the number of electrodes 58 and the biphasic waveform.

During the application of neurostimulation to the target tissue, alternating the leading phase of each successive pulse of the biphasic waveform stimulates a greater number of nerves within the target tissue as compared to traditional bipolar or monopolar stimulation. By stimulating a greater number of nerves within the target tissue, an optimal placement of the electrodes 58 within the target tissue for denervation can be more readily identified to ensure effective renal denervation and an optimal outcome. The location and/or orientation of the electrodes 58 relative to the tissue wall can be altered between the application of neurostimulation to map or otherwise identify optimal nerve candidates for denervation. In this manner, neurostimulation is applied to the target tissue via the electrodes 58 in a first orientation and a physiological response, such as an increase in blood pressure or an increase in vessel stiffness, is measured. As can be appreciated, stimulation of nerves within the renal artery can result in substantial blood pressure elevation and/or an increase in vessel wall stiffness. The measurements of these physiological responses to the neurostimulation may be compared to one or more thresholds, where a physiological response less than the one or more thresholds indicates that the stimulated nerves would not be good candidates for denervation whereas a physiological response greater than the one or more thresholds (e.g., blood pressure is greater and/or vessel stiffness is greater) indicates that the stimulated nerves would be good candidates for denervation. If the physiological response is less than the one or more thresholds (e.g., blood pressure is less and/or vessel stiffness is greater), the orientation of the electrodes 58 is changed by either advancing or retracting the therapeutic assembly 56 within the renal artery (e.g., proximally or distally) or rotating the therapeutic assembly 56 in a clockwise or anticlockwise direction.

Once the orientation of the electrodes 58 has been changed, the neurostimulation is once again applied to the target tissue and the physiological response is measured. Alternatively, the orientation of the electrodes 58 may be altered numerous times between the application of neurostimulation and the orientation having the greatest physiological response may be identified and utilized for the application of therapeutic or denervation energy. As can be appreciated, the above sequence may be repeated as many times as necessary to identify the optimal orientation and/or location of the electrodes 58 relative to the target tissue or to identify the optimal nerves for denervation.

It is envisioned that the physiological responses to the application of neurostimulation can be monitored by a control algorithm 44 stored on the computing device 20, with the location and results of the application of neurostimulation stored in the memory 32. The stored physiological responses can be compared to predetermined thresholds stored in the memory 32 or to other physiological responses stored in the memory to aid the clinician in identifying the optimal locations and/or orientation of the electrodes 58 relative to the target tissue. A look-up table of data of predetermined thresholds may be saved within the memory 32 and accessed by the control algorithm 44 or other suitable application stored on the memory 32 during the procedure and alerts may be presented on the user interface 28.

In addition to identifying candidates for a denervation procedure, it is envisioned that the biphasic neurostimulation waveform described herein may be utilized to analyze the efficacy of the denervation procedure. In this manner, after the application of therapy from the therapy source 24 to the target tissue, neurostimulation may be applied by the electrodes 58 to stimulate the nerves within the target tissue and measure a physiological response thereto. As can be appreciated, a successful denervation procedure would result in a reduced physiological response to the neurostimulation as compared to the physiological response to the neurostimulation applied before denervation. Similar to the procedure described hereinabove with respect to identifying potential candidates for denervation, the physiological response to the application of neurostimulation by the electrodes 58 can be measured and compared to one or more thresholds. In contrast to the above, a physiological response that is less than the one or more thresholds (e.g., blood pressure is lower and/or vessel stiffness is lower) is indicative of successful denervation whereas a physiological response that is greater than the one or more thresholds (e.g., blood pressure is higher and/or vessel stiffness is higher) suggests further denervation may be required. In embodiments where an increase in physiological response (e.g., heart rate) compared to one or more thresholds is a desired physiological response to denervation, a measured physiological response that is greater than a predetermined threshold is indicative of successful denervation whereas a physiological response that is less than the predetermined threshold suggests further denervation may be required.

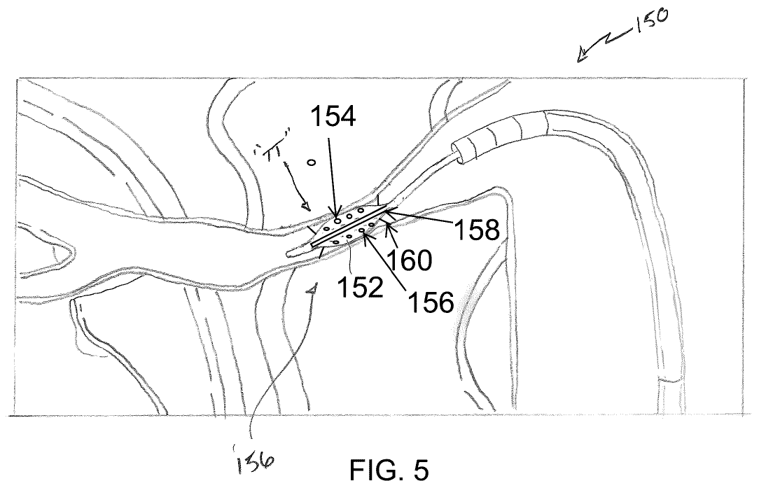

Heretofore, the therapeutic device 50 has been primarily described in connection with a shape memory construction where exit from a guide catheter 62 or withdrawal of a guidewire 64 frees the shape memory alloy to achieve a desired spiral shape of the therapeutic portion 56 of the therapeutic device 50. As noted elsewhere, however, the present disclosure is not so limited. With reference to FIG. 5, a therapeutic device 150 may employ a balloon 152. As hereinabove, during a navigation phase, the balloon 152 has a generally linear shape, and when at a desired location, the balloon 152 can be expanded to achieve a desired shape. The desired shape may be based on the side of the blood vessel or liminal tissue into which the therapeutic device 150 is to be navigated and to which therapy is to be applied. As is known in the art, balloons may be employed to ensure contact of RF electrodes 154 and ultrasound transducers 156 against tissue. Further, balloons may be employed to center a microwave antenna 158 in the blood vessel.

It is contemplated that the balloon 152 may be multi-chambered or have one or more fluid ports allowing, for example, for different mediums to flow into the balloon 152 or different chambers of the balloon 152. In one non-limiting embodiment, the balloon 152 may be inflated with saline that surrounds the microwave antenna 158 allowing for good coupling (e.g., impedance match) of the microwave energy emitted from the antenna to the tissue receiving tissue. The saline also works to sub-cool the tissue closest to the antenna (e.g., the blood vessel wall) to prevent necrosis of this tissue. The balloon 152 may also be used to limit the extent a cryogenic medium can act on tissues limiting the spread of the cryogenic medium to just the area of the balloon 152. In cryogenic and chemical ablations, the therapeutic device 150 may include one or more needles 160 that can selectively project (e.g., deploy) from the therapeutic device 150 and are fluidly coupled to a therapy source 24 supplying these therapies directly to the desired tissue. The balloon 152 in such cases provides a centered and stable platform for the needles 160 to exit the therapeutic deice 150 and enter the tissue. In with the present disclosure, the balloon 152 may be an occlusive balloon, a non-occlusive balloon, or another configuration of a balloon permitting the flow of blood or other media through the blood vessel of luminal tissue.

It is contemplated that the therapeutic device 50, 150 may include multiple modalities for therapy such that, for example, a single device therapeutic device may include RF electrodes 154 and ultrasound transducers 156, or RF electrodes 154 and chemical ablation needles 160. Each of these is connected to a therapy source 24 configured to supply the denoted therapy type. Those having skill in the art will recognize that the therapeutic device 150 and the therapy source 24 may provide any suitable combination of therapies capable of performing a denervation procedure.

Like the embodiments of FIGS. 3 and 4, the therapeutic device 150 may include RF electrodes 154 disposed thereon for the delivery of therapy and/or neurostimulation to the tissue as described hereinabove. Where, for example, the therapy modality is not RF ablation, and thus there are not electrodes 154 on the balloon the guide catheter 62 may include two or more electrodes 66 (FIG. 4) disposed thereon for delivery of the neurostimulation to the desired tissue. The electrodes 66 of the guide catheter 62 are utilized to deliver the biphasic neurostimulation generated by the neurostimulation source 24a to the target tissue and identify nerves that would be a good candidate for denervation, as described in further detail hereinabove. With candidate nerves identified and the position at which the therapeutic device 50/150 should be located identified, the guide catheter 62 may be retracted (as shown in FIG. 5) to uncover the therapeutic portion 156 and balloon 152, to allow for application of therapy via the RF electrodes 154 (if employed), ultrasound transducers 156, microwave antenna 158, and/or needles 160.

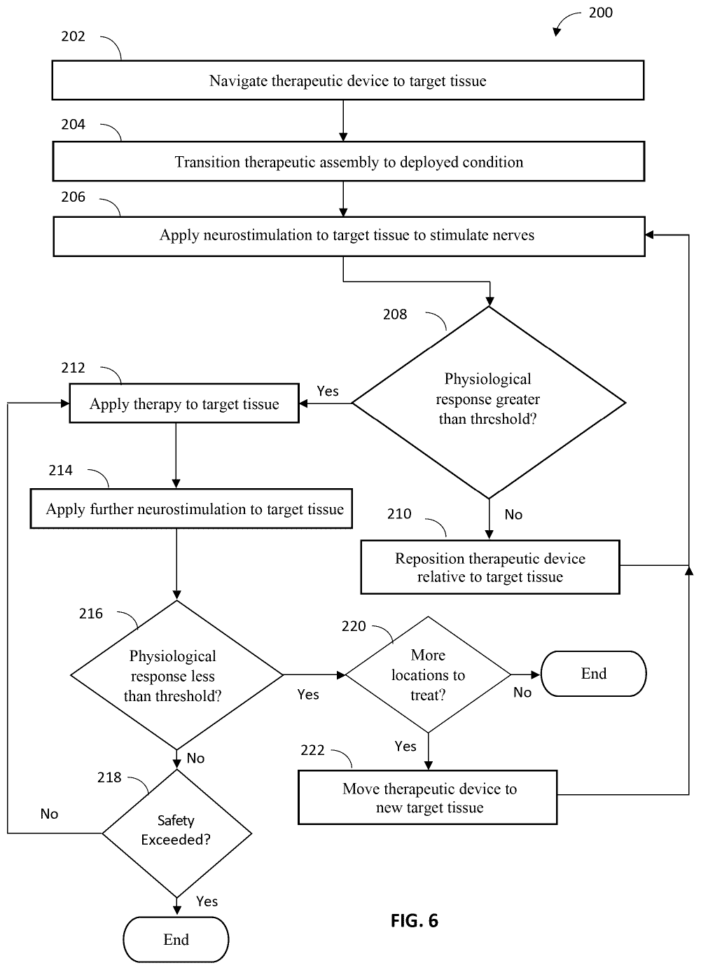

Turning to FIG. 6, a method of performing a therapeutic procedure is illustrated and generally identified by reference numeral 200. In step 202, the therapeutic device 50 is navigated to the target tissue. Once the therapeutic device is located adjacent the target tissue, the therapeutic portion may optionally be transitioned from a first state to a second deployed state in step 204 such that the one or more therapy delivery elements abut or otherwise contact the tissue. Step 204 may alternatively occur after step 208 in instances where the neurostimulation electrodes 66 are on a guide catheter 62, guidewire 64, or another device as depicted in FIG. 4. In step 206, neurostimulation is applied to the blood vessel or luminal tissue to effectuate stimulation of the nerves within the tissue. In step 208, a physiological response to the application of neurostimulation to the blood vessel or luminal tissue is monitored and/or observed and compared against a predetermined threshold. If the physiological response to the neurostimulation is less than the predetermined threshold, the therapeutic device is repositioned relative to the blood vessel or luminal tissue in step 210, and thereafter, the method returns to step 206. If the physiological response to the neurostimulation is greater than the predetermined threshold, therapy is applied to the blood vessel or luminal tissue in step 212. After the application of therapy to the blood vessel or luminal tissue, further neurostimulation is applied to the blood vessel or luminal tissue in step 214 and the physiological response to the application of neurostimulation is monitored and/or observed and compared to a predetermined threshold in step 216. If the physiological response to the neurostimulation was greater than the threshold, an indication of incomplete denervation, the method moves to step 218 where a check is made to ensure that no safety parameter has been exceeded. The safety parameter may be, for example, an assessment of a total duration of the procedure, the total energy delivered or removed from the tissue, or other parameters which if exceeded could undesirably damage tissues proximate the target tissue. If any of these have been exceeded, the method ends. If no safety parameters has been exceeded at step 218, the method returns to step 212 for further application of therapy. In this manner, therapy can be applied until successful denervation has been achieved. At step 216, if the physiological response to the neurostimulation is less than the threshold, a determination is made whether additional treatment locations remain to receive therapy in step 220. If no, the method ends, however, if one or more treatment sites remain to receive therapy the therapeutic device is moved to one of those locations in step 222 and the process returns to step 206 for the application of neurostimulation to the target tissue at the new treatment site. The method continues until all tissue sites have received the desired therapy and show evidence of successful denervation.

Although described generally hereinabove, it is envisioned that the memory 32 may include any non-transitory computer-readable storage media for storing data and/or software including instructions that are executable by the processor 30 and which control the operation of the workstation 20 and, in some embodiments, may also control the operation of the therapeutic device 50, imaging device 70, and/or ECG machine. In an embodiment, memory 32 may include one or more storage devices such as solid-state storage devices, e.g., flash memory chips. Alternatively, or in addition to the one or more solid-state storage devices, the memory 32 may include one or more mass storage devices connected to the processor 30 through a mass storage controller (not shown) and a communications bus (not shown).

Although the description of computer-readable media contained herein refers to solid-state storage, it should be appreciated by those skilled in the art that computer-readable storage media can be any available media that can be accessed by the processor 30. That is, computer readable storage media may include non-transitory, volatile, and non-volatile, removable, and non-removable media implemented in any method or technology for storage of information such as computer-readable instructions, data structures, program modules or other data. For example, computer-readable storage media may include RAM, ROM, EPROM, EEPROM, flash memory or other solid-state memory technology, CD-ROM, DVD, Blu-Ray or other optical storage, magnetic cassettes, magnetic tape, magnetic disk storage or other magnetic storage devices, or any other medium which may be used to store the desired information, and which may be accessed by the energy source 20.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of embodiments. Those skilled in the art will envision other modifications within the scope and spirit of the claims appended hereto.

Further disclosed herein is the subject-matter of the following clauses:

1. A method of performing and assessing a therapeutic procedure, comprising:

navigating a therapeutic device to target tissue;

transitioning the therapeutic device from a first, linear configuration to a second, helical configuration such that a plurality of electrodes on the therapeutic device are in engagement with the target tissue;

applying pulses of neurostimulation energy having at least two phases to the target tissue via the plurality of electrodes, wherein each pulse of the neurostimulation energy includes an anodal phase and a cathodal phase, and a phase of the neurostimulation energy is switched from anodal to cathodal or cathodal to anodal for each successive pulse;

observing a physiological response to the pulses of neurostimulation energy in excess of a threshold and indicative of a neural response;

denervating the nerves of the target tissue; and

applying pulses of the neurostimulation energy to the target tissue, wherein a physiological response less than a threshold is indicative of a successful denervation of the nerves of the target tissue.

2. The method according to clause 1, wherein the physiological response is blood pressure or vessel stiffness.

3. The method according to clause 1, wherein the target tissue is one or more of a renal artery, a splanchnic artery, or a hepatic artery.

4. The method according to clause 1, wherein during the anodal phase energy is applied to a first of the plurality of electrodes and received by a second of the plurality of electrodes.

5. The method according to clause 4, wherein during the cathodal phase energy is applied to the second of the plurality of electrodes and received by the first of the plurality of electrodes.

6. The method according to clause 4, wherein during the anodal phase or cathodal phase energy is applied to two or more of the plurality of electrodes or received by two or more of the plurality of electrodes.

7. The method according to clause 1, wherein the denervation is achieved by application of monopolar energy to the target tissue via the plurality of electrodes.

8. The method according to clause 1, wherein the denervation is achieved by a therapy modality selected from the group consisting of radiofrequency ablation, microwave ablation, ultrasound ablation, cryoablation, and chemical ablation.

9. The method according to clause 1, wherein the neurostimulation energy includes a frequency of between about 10-30 Hz, a pulse width of between about 2-10 ms, a voltage of between about 5-30 V, and a current of between about 2-500 mA.

10. The method according to clause 1, wherein the neurostimulation energy is multiphasic.

11. The method according to clause 10, wherein the neurostimulation energy is biphasic or triphasic.

12. A method of assessing a target location for therapy, comprising:

applying pulses of neurostimulation to tissue at a target location wherein the neurostimulation pulses include an anodal phase and a cathodal phase;

switching a phase of the neurostimulation pulses from anodal to cathodal or cathodal to anodal for each successive pulse; and

observing a physiological response to the neurostimulation pulses, wherein a response in excess of a threshold is indicative of a neural response signifying the target location is a candidate for application of a therapy.

13. The method according to clause 12, wherein the physiological response is blood pressure or vessel stiffness.

14. The method according to clause 12, wherein the target location is one or more of a renal artery, a splanchnic artery, or a hepatic artery.

15. The method according to clause 12, wherein during the anodal phase neurostimulation energy is applied to a first of a plurality of electrodes and received by a second of the plurality of electrodes.

16. The method according to clause 15, wherein during the cathodal phase neurostimulation energy is applied to the second of the plurality of electrodes and received by the first of the plurality of electrodes.

17. The method according to clause 16, wherein during the anodal phase or cathodal phase neurostimulation energy is applied to two or more of the plurality of electrodes or received by two or more of the plurality of electrodes.

18. The method according to clause 12, further comprising navigating a diagnostic device to the target location.

19. The method according to clause 18, further comprising transitioning the diagnostic device from a first configuration to a second configuration such that a plurality of electrodes on the diagnostic device are in engagement with tissue at the target location.

20. The method according to clause 19, wherein the diagnostic device transitions from a linear configuration to a helical configuration to place the electrodes in engagement with tissue at the target location.

21. The method according to clause 20, further comprising a balloon to place the electrodes in engagement with tissue at the target location.

22. The method according to clause 18, wherein the diagnostic device is a guide catheter or a guidewire having a plurality of electrodes disposed thereon for the delivery of the neurostimulation to the target tissue.

23. The method according to clause 22, further comprising placing at least two of the plurality of electrodes of the guide catheter or guidewire into engagement with tissue at the target location.

24. The method according to clause 12, further comprising applying denervation therapy to the target tissue, the denervation therapy selected from the group consisting of radiofrequency ablation, microwave ablation, ultrasound ablation, cryoablation, and chemical ablation.

25. The method according to clause 12, wherein the neurostimulation energy is multiphasic.

26. The method according to clause 25, wherein the neurostimulation energy is biphasic or triphasic.

27. A system for performing a diagnostic and therapeutic procedure, comprising:

a catheter including a plurality of electrodes and configured for placement proximate target tissue; and

an energy source operably coupled to the catheter, the energy source having:
a diagnostic mode, wherein pulses of neurostimulation energy are generated for delivery between at least two of the plurality of electrodes in a bipolar manner, the pulsed neurostimulation energy including an anodal phase and a cathodal phase, and the energy source configured to switch the phases of the pulsed neurostimulation energy from anodal to cathodal or cathodal to anodal for each successive pulse; and
a denervation mode, wherein monopolar radio-frequency energy is generated for delivery by the plurality of electrodes for denervation of nerves of the target tissue.

28. The system according to clause 27, wherein the catheter has a first configuration and a second configuration, wherein in the second configuration the plurality of electrodes on the catheter are in engagement with tissue at the target tissue.

29. The system according to clause 28, wherein the first configuration is a linear configuration for navigation to the target tissue and the second configuration is a helical configuration to place the plurality of electrodes in engagement with tissue at the target tissue.

30. The system according to clause 28, further comprising a balloon disposed on the catheter, wherein inflation of the balloon places the plurality of electrodes in engagement with the target tissue.

31. A system for performing a diagnostic and therapeutic procedure, comprising:

a catheter including a plurality of electrodes and configured for placement proximate target tissue;

an energy source operably coupled to the catheter, the energy source generating pulses of neurostimulation energy for delivery between at least two of the plurality of electrodes in a bipolar manner, the pulsed neurostimulation energy including an anodal phase and a cathodal phase, and the energy source configured to switch a phase of the pulsed neurostimulation energy from anodal to cathodal or cathodal to anodal for each successive pulse; and

a therapy source coupled to the catheter for delivery of denervation therapy for denervation of nerves of the target tissue.

32. The system according to clause 31, wherein the therapy source is selected from the group consisting of a cryogenic source for delivery of cryogenic medium, an RF generator for generating monopolar radio-frequency energy, a microwave generator for generating microwave energy, and a chemical source for delivery of a chemical medium.

33. The system according to clause 31, further comprising a balloon disposed on the catheter, wherein inflation of the balloon places the plurality of electrodes in engagement with tissue at the target tissue.

34. The system according to clause 33, wherein the therapy source is a cryogenic source for delivery of cryoablation medium, wherein the balloon is inflated with the cryoablation medium.

35. The system according to clause 31, wherein the energy source is integrated with the therapy source.

36. The system according to clause 31, wherein the catheter includes a second plurality of electrodes configured for placement proximate target tissue, the second plurality of electrodes coupled to the therapy source for delivery of denervation therapy to the target tissue.

37. The system according to clause 31, wherein the energy source and the therapy source are each coupled to the plurality of electrodes, such that the plurality of electrodes delivery neurostimulation energy to the tissue in a diagnostic mode and denervation therapy to the tissue in a denervation mode.

38. The system according to clause 31, wherein the catheter has a first configuration and a second configuration, wherein in the second configuration the plurality of electrodes on the catheter are in engagement with tissue at the target tissue.

39. The system according to clause 38, wherein the first configuration is a linear configuration for navigation to the target tissue and the second configuration is a helical configuration to place the plurality of electrodes in engagement with tissue at the target tissue.

40. A system for performing a diagnostic and therapeutic procedure, comprising:

a catheter including a plurality of electrodes and configured for placement proximate target tissue; and

a workstation operably coupled to the catheter, the workstation including a memory and a processor, the memory storing instructions, which when executed by the processor cause the processor to:
apply pulses of neurostimulation energy having at least two phases to the target tissue via the plurality of electrodes, wherein each pulse of the neurostimulation energy includes an anodal phase and a cathodal phase, and a phase of the neurostimulation energy is switched from anodal to cathodal or cathodal to anodal for each successive pulse;
observe a physiological response to the pulses of neurostimulation energy in excess of a threshold and indicative of a neural response;
denervate the nerves of the target tissue; and
apply pulses of the neurostimulation energy to the target tissue, wherein a physiological response less than a threshold is indicative of a successful denervation of the nerves of the target tissue.

41. The system according to clause 40, further comprising an energy source operably coupled to the catheter, the energy source configured to generate a therapy modality selected from the group consisting of radiofrequency ablation, microwave ablation, ultrasound ablation, cryoablation, and chemical ablation.

42. The system according to clause 41, wherein the energy source is configured to generate the neurostimulation energy when in a diagnostic mode and generate a therapy modality when in a denervation mode.

43. The system according to clause 40, wherein the neurostimulation energy includes a frequency of between about 10-30 Hz, a pulse width of between about 2-10 ms, a voltage of between about 5-30 V, and a current of between about 2-500 mA.

44. The system according to clause 40, further comprising a balloon disposed on the catheter, wherein inflation of the balloon places the plurality of electrodes in engagement with the target tissue.

1. A system for performing a diagnostic and therapeutic procedure, comprising:
a catheter including a plurality of electrodes and configured for placement proximate target tissue; and
an energy source operably coupled to the catheter, the energy source having:
a diagnostic mode, wherein pulses of neurostimulation energy are generated for delivery between at least two of the plurality of electrodes in a bipolar manner, the pulsed neurostimulation energy including an anodal phase and a cathodal phase, and the energy source configured to switch the phases of the pulsed neurostimulation energy from anodal to cathodal or cathodal to anodal for each successive pulse; and
a denervation mode, wherein monopolar radio-frequency energy is generated for delivery by the plurality of electrodes for denervation of nerves of the target tissue.

2. The system according to claim 1, wherein the catheter has a first configuration and a second configuration, wherein in the second configuration the plurality of electrodes on the catheter are in engagement with tissue at the target tissue.

3. The system according to claim 2, wherein the first configuration is a linear configuration for navigation to the target tissue and the second configuration is a helical configuration to place the plurality of electrodes in engagement with tissue at the target tissue.

4. The system according to claim 2, further comprising a balloon disposed on the catheter, wherein inflation of the balloon places the plurality of electrodes in engagement with the target tissue.

5. A system for performing a diagnostic and therapeutic procedure, comprising:
a catheter including a plurality of electrodes and configured for placement proximate target tissue;
an energy source operably coupled to the catheter, the energy source generating pulses of neurostimulation energy for delivery between at least two of the plurality of electrodes in a bipolar manner, the pulsed neurostimulation energy including an anodal phase and a cathodal phase, and the energy source configured to switch a phase of the pulsed neurostimulation energy from anodal to cathodal or cathodal to anodal for each successive pulse; and
a therapy source coupled to the catheter for delivery of denervation therapy for denervation of nerves of the target tissue.

6. The system according to claim 5, wherein the therapy source is selected from the group consisting of a cryogenic source for delivery of cryogenic medium, an RF generator for generating monopolar radio-frequency energy, a microwave generator for generating microwave energy, and a chemical source for delivery of a chemical medium.

7. The system according to claim 5, further comprising a balloon disposed on the catheter, wherein inflation of the balloon places the plurality of electrodes in engagement with tissue at the target tissue.

8. The system according to claim 7, wherein the therapy source is a cryogenic source for delivery of cryoablation medium, wherein the balloon is inflated with the cryoablation medium.

9. The system according to claim 5, wherein the energy source is integrated with the therapy source.

10. The system according to claim 5, wherein the catheter includes a second plurality of electrodes configured for placement proximate target tissue, the second plurality of electrodes coupled to the therapy source for delivery of denervation therapy to the target tissue.

11. The system according to claim 5, wherein the energy source and the therapy source are each coupled to the plurality of electrodes, such that the plurality of electrodes delivery neurostimulation energy to the tissue in a diagnostic mode and denervation therapy to the tissue in a denervation mode.

12. The system according to claim 5, wherein the catheter has a first configuration and a second configuration, wherein in the second configuration the plurality of electrodes on the catheter are in engagement with tissue at the target tissue.

13. The system according to claim 12, wherein the first configuration is a linear configuration for navigation to the target tissue and the second configuration is a helical configuration to place the plurality of electrodes in engagement with tissue at the target tissue.

14. A system for performing a diagnostic and therapeutic procedure, comprising:
a catheter including a plurality of electrodes and configured for placement proximate target tissue; and
a workstation operably coupled to the catheter, the workstation including a memory and a processor, the memory storing instructions, which when executed by the processor cause the processor to:
apply pulses of neurostimulation energy having at least two phases to the target tissue via the plurality of electrodes, wherein each pulse of the neurostimulation energy includes an anodal phase and a cathodal phase, and a phase of the neurostimulation energy is switched from anodal to cathodal or cathodal to anodal for each successive pulse;
observe a physiological response to the pulses of neurostimulation energy in excess of a threshold and indicative of a neural response;
denervate the nerves of the target tissue; and
apply pulses of the neurostimulation energy to the target tissue, wherein a physiological response less than a threshold is indicative of a successful denervation of the nerves of the target tissue.

15. The system according to claim 14, further comprising an energy source operably coupled to the catheter, the energy source configured to generate a therapy modality selected from the group consisting of radiofrequency ablation, microwave ablation, ultrasound ablation, cryoablation, and chemical ablation.

16. The system according to claim 15, wherein the energy source is configured to generate the neurostimulation energy when in a diagnostic mode and generate a therapy modality when in a denervation mode.

17. The system according to claim 14, wherein the neurostimulation energy includes a frequency of between about 10-30 Hz, a pulse width of between about 2-10 ms, a voltage of between about 5-30 V, and a current of between about 2-500 mA.

18. The system according to claim 14, further comprising a balloon disposed on the catheter, wherein inflation of the balloon places the plurality of electrodes in engagement with the target tissue.
